(19) United States
(12) Patent Application Publication
(10) Pub. No.: US 2017/0105870 A1
(43) Pub. Date: Apr. 20, 2017

(54) FLUID-BASED THERMAL CLOTHING FOR HEAT EXCHANGE WITH BODY PARTS

(71) Applicant: Ataollah Yazdani, Beaverton, OR (US)

(72) Inventor: Ataollah Yazdani, Beaverton, OR (US)

(21) Appl. No.: 14/883,505

(22) Filed: Oct. 14, 2015

(51) Int. Cl. A61F 7/02; A41D 13/005; A43B 3/00; A43B 1/00; A43B 3/02; A43B 7/12

(52) U.S. Cl. CPC A61F 7/02; A43B 3/02; A43B 7/12; A43B 3/0005; A43B 1/0072; A41D 13/0051; A41D 13/0053; A61F 2007/0045

(57) ABSTRACT

A method and an article of manufacture are disclosed for exchanging heat with a human body part to reduce fever, reduce localized swelling, warm up feet, cool down the body, increase body core temperature, and the like. A double walled water-tight article of clothing, such as a boot or a glove, may include a fluid inlet and outlet. A single walled but water-tight article of clothing may be used around a body part and allow the water to contact the skin inside the article of clothing for more rapid and direct heat exchange. Pumps, heating or cooling elements, thermometers or other devices may be included with the article of clothing. The article of clothing may be compartmentalized to concentrate the fluid around a particular body part. The fluid may continuously enter through an inlet and exit from an outlet in the article of clothing, or be a fixed amount.

### TECHNICAL FIELD

This application relates generally to thermal clothing. More specifically, this application relates to a method and apparatus for altering body temperature by exchanging heat between a circulating fluid and a body part like a leg or an arm.

### BRIEF DESCRIPTION OF THE DRAWINGS

The drawings, when considered in connection with the following description, are presented for the purpose of facilitating an understanding of the subject matter sought to be protected.

FIG. 1A shows an example fluid-based heat exchanging boot usable to cool or heat lower leg and foot;

FIG. 1B shows an example fluid-based heat exchanging glove and elbow cover usable to cool or heat elbow, wrist, or hand and fingers;

FIG. 2A shows an example direct-contact fluid-based heat exchanging boot usable to cool or heat lower leg and foot;

FIG. 2B shows an example direct-contact fluid-based heat exchanging glove and elbow cover usable to cool or heat elbow, wrist, or hand and fingers;

FIG. 3 shows an example fluid-based heat exchanging boot with entry and exist thermometers, fluid pump, and display usable to cool or heat lower leg and foot;

FIG. 4 shows an example fluid-based heat exchanging boot with active heating and/or cooling elements usable to cool or heat lower leg and foot; and

FIG. 5 shows an example compartmentalized fluid-based heat exchanging boot usable to cool or heat targeted body parts.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while the following description references boots and gloves, it will be appreciated that the disclosure may include other heat exchanging articles of clothing such as vest, knee band or strap, waist band, shoulder strap, neck or head cover, and the like.

Briefly described, a method and an article of manufacture are disclosed for exchanging heat (either warming or cooling) with a human body part to reduce fever, reduce localized swelling due to trauma or other causes, warm up feet for sleeping, cool down overall body temperature to prevent heat stroke, increase body core temperature, and the like. In various embodiments a double walled water-tight article of clothing, such as a boot or a glove, may include a fluid inlet and outlet to allow water to move through the space between the walls while exchanging heat with the body part wearing the article of clothing. In other embodiments, a single walled but water-tight article of clothing may be used around a body part and allow the water to contact the skin inside the article of clothing for more rapid and direct heat exchange. In some embodiments, pumps, heating or cooling elements, thermometers or other devices may be included with the article of clothing, whether integrated or attached to article of clothing, to provide other appropriate functions like circulating the water, heating or cooling the water, and measuring the water temperature, respectively. In still other embodiments, the article of clothing may be compartmentalized to direct and concentrate the fluid around a particular body part, such as the wrist in a double-walled or single-walled watertight glove. In some embodiments, the fluid may continuously enter through an inlet and exit from an outlet in the article of clothing, while in other embodiments, the amount of fluid may be constant and contained within the article of clothing but be circulated to exchange heat between the body part and heat sources or sinks, such as fins or heaters/coolers, integrated with the article of clothing.

Body temperature in humans plays an important role in general health, the functioning of organs, muscles, and joints, and comfort. The human body core temperature is nominally about 98.6 degrees Fahrenheit or 37 degrees Celsius. The temperature at extremities, such as arms and legs, hands and feet, and head and neck may vary due to exposure to heat or cold, perspiration, contact with water and flowing air (wind), exposure to night sky and other situations in which heat is added or removed from the body. The addition or removal of heat from skin surface, especially near extremities, is one way the nervous system regulates the core temperature. This is generally done by directing blood to or away from exposed skin for more or less heat exchange, respectively. Other methods of adding heat and raising the temperature of the body include metabolization of food and shivering. People can actively aid in their body's temperature regulation by fanning themselves, seeking shade or sun, using heating or cooling devices like heaters and air conditioners, varying the layers and types of clothing they wear, immersion in water, and the like.

The above methods of regulating body temperature to comfortable levels are generally for normal and healthy conditions. However, at times, the body temperature may drop or rise in response to abnormal health conditions such as fever, trauma (especially to joints and muscles), or poor blood circulation due to age, neurological problems, or other medical causes. In such cases extra help or measures to add or remove heat from the body may be needed. Sometimes the exchange of heat (either removing or adding) may be for the benefit of the entire body, as in the case of high fever, while at other times heat exchange may be needed for localized sites on the body such as reducing the swelling of a traumatized joint like the knee or elbow.

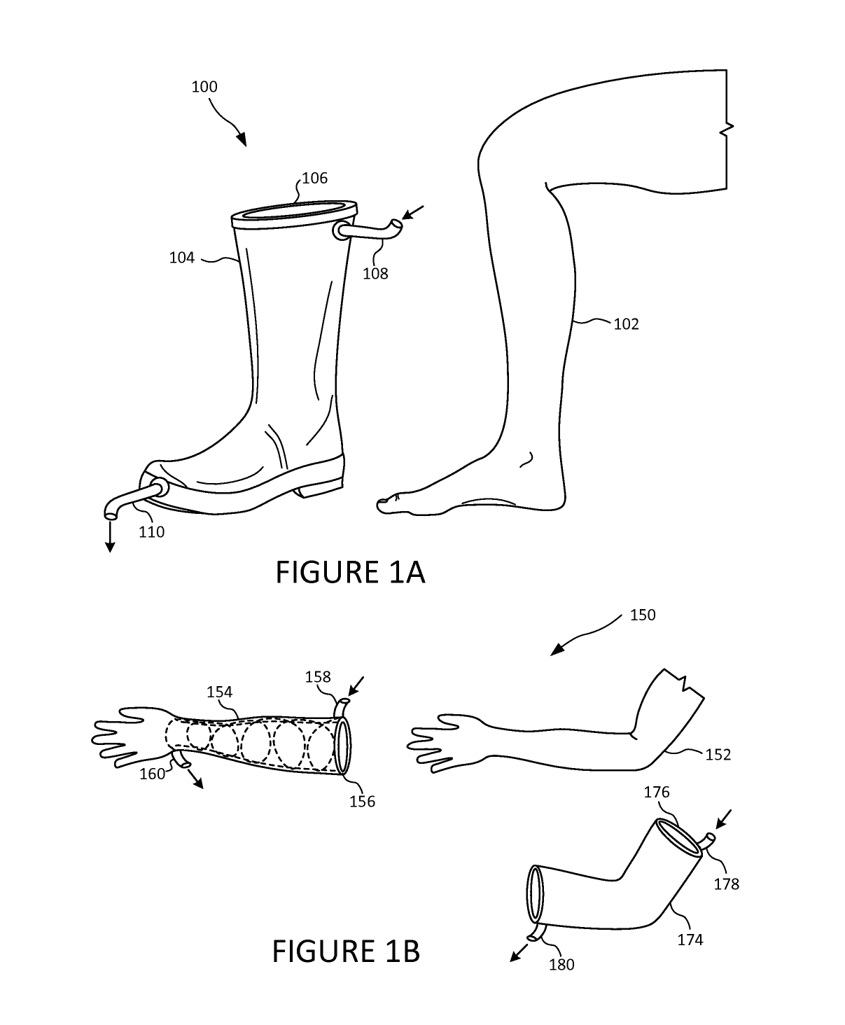

FIG. 1A shows an example fluid-based heat exchanging boot usable to cool or heat lower leg and foot. In various embodiments, heat exchange apparatus 100 includes a double-walled boot 104 to receive a human leg and foot 102. The boot includes double-walled construction creating a water-tight inter-wall space 106 to contain water or other heat exchange fluid such as alcohol entering the inter-wall space or fluid enclosure through fluid inlet 108 and exiting through fluid outlet 110.

In various embodiments, the double-walled boot may be made of plastic to be waterproof. In other embodiments, the boot may be made of any other material such as leather, cloth, nylon, or any other suitable material with a waterproof liner inserted within the fluid chamber or enclosure space 106 to contain the fluid.

In various embodiments, the fluid inlet 108 and outlet 110 may be a small hose with a valve at the base to open or close fluid path or control the flow rate of the fluid through the fluid chamber. In some embodiments, the fluid inlet and outlet are the same, so that fluid may be added or removed from the same single opening.

In various embodiments, the fluid may be water, alcohol, air, or other suitable heat exchange fluid medium.

In various embodiments, to cool or warm the foot or leg, the boot may be worn and then connected to a source of heat exchange fluid, such as tap water from a faucet or a reservoir of cold or warm water. On the drain side, as the water enters the inlet, it can exit the outlet to keep fresh water at a desired and controlled temperature circulating around the body part inside the boot. In some embodiments, ridges, grooves, or similar pathways may be provided inside the boot where the water flows to guide the flow of water and control it to flow or circulate in a spiral, up-and-down, diagonal, or in other flow patterns around the body part being treated. In other embodiments, the water may poured into the boot from inlet 108 at a certain temperature without opening the drain 110 to fill the boot and allow the water to stay without flow to more slowly cool or warm the body part compared to flowing water which creates convection and more rapid heat exchange. In some embodiments, the speed of the flow may be controlled from inlet 108 to outlet 110 by partially opening the inlet and/or the outlet.

In some embodiments, the boot may be made of transparent plastic or rubber to allow viewing the water circulating around the target body part such as leg and foot. Other devices may be used to make the boot or other heat-exchanging article of clothing more interesting and entertaining to children, such as pictures or blinking LED (Light Emitting Diode) lights attached to surface of boot, floating shaped particles or swaying color bands inside the water enclosure, and the like. In various embodiments, the floating ornamental particles may be in the shape of colorful fish, submarines, ship, mermaids, and the like to entertain small children.

The circulation of cold fluid around the target body part can help reduce fever or subside swelling, while the circulation of warm or hot water can help heat a joint to reduce pain or stiffness or relieve chill. This arrangement may be especially interesting for children who often have aversion to medications or other medical treatments. Children having high fever may feel better by putting their feet in cool water to reduce their core temperature, while also being amused by watching the water circulate and the floats move around.

FIG. 1B shows an example fluid-based heat exchanging glove and elbow cover usable to cool or heat elbow, wrist, or hand and fingers. In various embodiments, heat exchange apparatus 150 includes a double-walled glove 154 to receive a human arm and hand 152. The glove includes double-walled construction creating an inter-wall space 156 to contain water or other heat exchange fluid such as alcohol entering the inter-wall space through fluid inlet 158 and exiting through fluid outlet 160. Similarly, a double-walled elbow brace or wrap 174 to receive a human elbow. The elbow brace includes double-walled construction creating an inter-wall space 176 to contain water or other heat exchange fluid such as alcohol entering the inter-wall space through fluid inlet 178 and exiting through fluid outlet 180.

In various embodiments, to cool or warm the arm or hand, the glove or elbow brace may be worn and then connected to a source of heat exchange fluid, such as tap water or a reservoir of cold or warm water. On the drain side, as the water enters the inlet, it can exit the outlet to keep fresh water at a desired and controlled temperature circulating around the body part inside the glove or elbow brace. In some embodiments, ridges, grooves, or similar pathways may be provided inside the heat-exchanging article of clothing where the water flows to guide the flow of water and control it to flow or circulate in a spiral, up-and-down, diagonal, or in other flow patterns around the body part being treated, as depicted by the dotted spiral line in glove 154. In other embodiments, the water may poured into the glove or elbow brace from inlet 158 or 178 at a certain temperature without opening the drain 160 or 180 to fill the article of clothing and allow the water to stay without flow to more slowly cool or warm the body part compared to flowing water which creates convection and more rapid heat exchange. In some embodiments, the speed of the flow may be controlled from inlet to outlet by partially opening the inlet and/or the outlet.

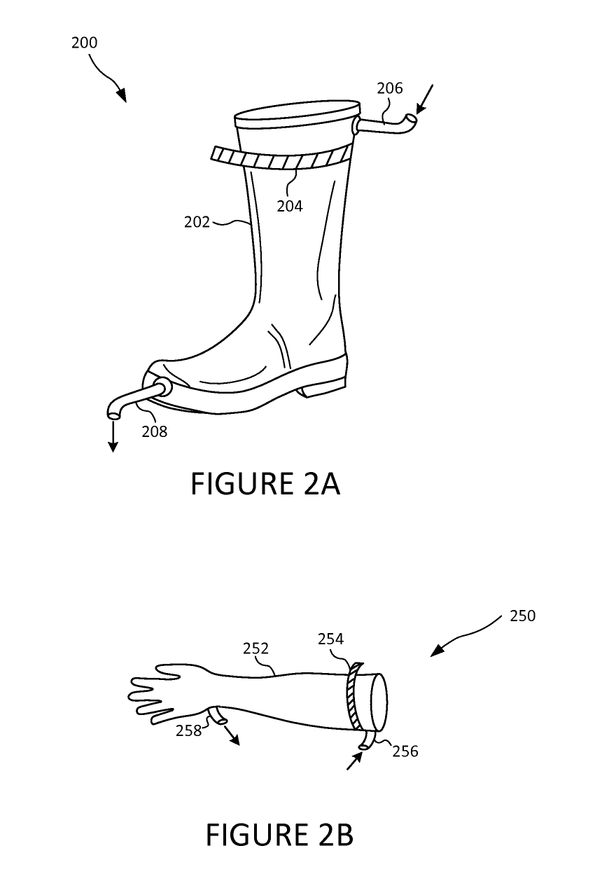

FIG. 2A shows an example direct-contact fluid-based heat exchanging boot usable to cool or heat lower leg and foot. In various embodiments, heat exchange apparatus 200 includes a single-walled boot 202 to receive a human leg and foot. The boot includes single-walled construction creating an inner space between the boot and the leg enclosed in it to contain water or other heat exchange fluid such as alcohol entering the inner space through fluid inlet 206 and exiting through fluid outlet 208. The boot may include a belt, band, hook and loop fastener 204, or other similar and suitable fastening device to make the top of the boot water tight to prevent the cooling/heating fluid from spilling back out of the boot.

In various embodiments, direct contact of the heat exchange fluid with skin allows faster heat transfer and thus, heating or cooling effect. The fluid may be poured directly into the boot or through the inlet 204 provided for this purpose. Once the fluid is poured in, the fastener 204 is fastened to create a closed enclosure with a tight seal between the boot and the leg, thus preventing the cooling fluid from spilling out. Other characteristics of the boot, such as flow control and spiral circulation and others described above, may be similar to the double-walled boot.

FIG. 2B shows an example direct-contact fluid-based heat exchanging glove and elbow cover usable to cool or heat elbow, wrist, or hand and fingers. In various embodiments, heat exchange apparatus 250 includes a single-walled glove 252 to receive a human arm and hand. The glove includes single-walled construction creating an inner space between the glove and the arm enclosed in it to contain water or other heat exchange fluid such as alcohol entering the inner space through fluid inlet 256 and exiting through fluid outlet 258. The glove may include a belt, band, hook and loop fastener 254, or other similar and suitable fastening device to make the top (entry point for hand) of the glove water tight to prevent the cooling/heating fluid from spilling back out of the glove. In other respects, the glove operates similarly to the boot.

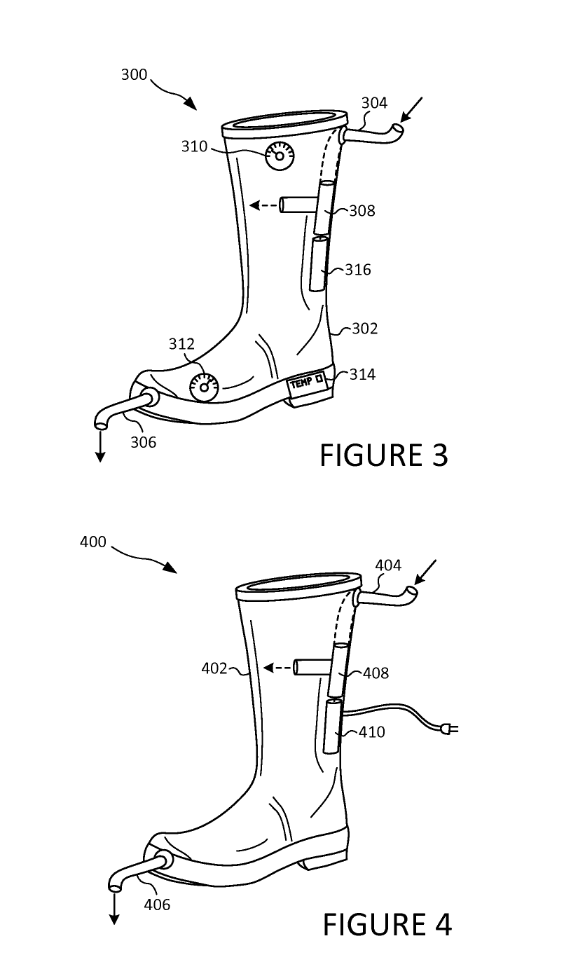

FIG. 3 shows an example fluid-based heat exchanging boot with entry and exist thermometers, fluid pump, and display usable to cool or heat lower leg and foot. In various embodiments, the heat exchange apparatus 300 includes double-walled or single-walled boot 302, fluid inlet 304, fluid drain or outlet 306, pump 308, inlet thermometer 310, outlet thermometer 312, processing unit with information display screen 314 having various buttons for control of computations and display, and battery pack 316.

In various embodiments, the heat-exchanging article of clothing, such as boot 302, includes entry and exit thermometers 310 and 312, respectively, to measure and display the temperature of the fluid entering and existing. This information may be useful in controlling flow rate, fluid temperature, and effectiveness of heating and cooling of the body part, such as leg and foot. In various embodiments, the data from thermometers may be transmitted, by wire or wirelessly, to the processor or processing unit 314, to calculate secondary parameters such as cooling or heating rate, thermal calories imparted or removed from body, estimated body temperature, and the like.

In various embodiments, the pump 308 may be used to circulate the water inside the fluid enclosure of the boot without opening the drain 306 or the inlet 304. In this configuration, once the fluid enclosure of the boot is filled with sufficient amount of water or other fluid, then inlet and outlet may be closed off and the pump turned on to circulate the fluid in a closed system and enhance thermal exchange with the target body part, such as foot or ankle, via convection. The inlet 304 and outlet 306 may also be connected together to create a closed fluid circuit in which water is circulated and no water is added or drained. This arrangement allows the user to step away from a water source, such as a faucet, and also to save water. In some embodiments, the power to operate the pump is provided by the battery pack 316, while in other embodiments an external power source, such as external battery pack or AC (Alternating Current) plug may be used.

In various embodiments, processing unit 314 may include a processor for computation and control of data and signals from other sources in the boot, such as thermometers, the display screen, the pump, and the heater/cooler. Such processing unit may further include some memory for program and data, data and control signal buses, input/output ports, and other electronic devices as needed, such as beepers and LED lights for status information and alarm.

FIG. 4 shows an example fluid-based heat exchanging boot with active heating and/or cooling elements usable to cool or heat lower leg and foot. In various embodiments, the heat exchange apparatus 400 includes double-walled or single-walled boot 402, fluid inlet 404, fluid drain or outlet 406, heating and/or cooling device 308, and power source 410.

In various embodiments, the heating and/or cooling device 408 may include a simple heating element to heat up the fluid entering the boot through the heater so that if water is provided from a cool source, it can be heated to a desired temperature. Similarly, water entering the boot may be cooled by an electronic cooling device such as a thermoelectric cooler by first passing the water through the cooler at the entry of the fluid enclosure of the boot. In some embodiments, the heating/cooling device may be included in a thermal unit which also includes a battery for the purpose of heating/cooling the fluid. In various embodiments, once the fluid enclosure of the boot is filled with sufficient amount of water or other fluid, then inlet and outlet may be closed off and the heater/cooler and/or the pump turned on to change the temperature of the fluid to a value different form the target body part and also to circulate the fluid and enhance thermal exchange with the target body part, such as foot or ankle, via convection, if desired.

In various embodiments, the power source 410 may be a battery pack, a rechargeable battery, or an AC adapter.

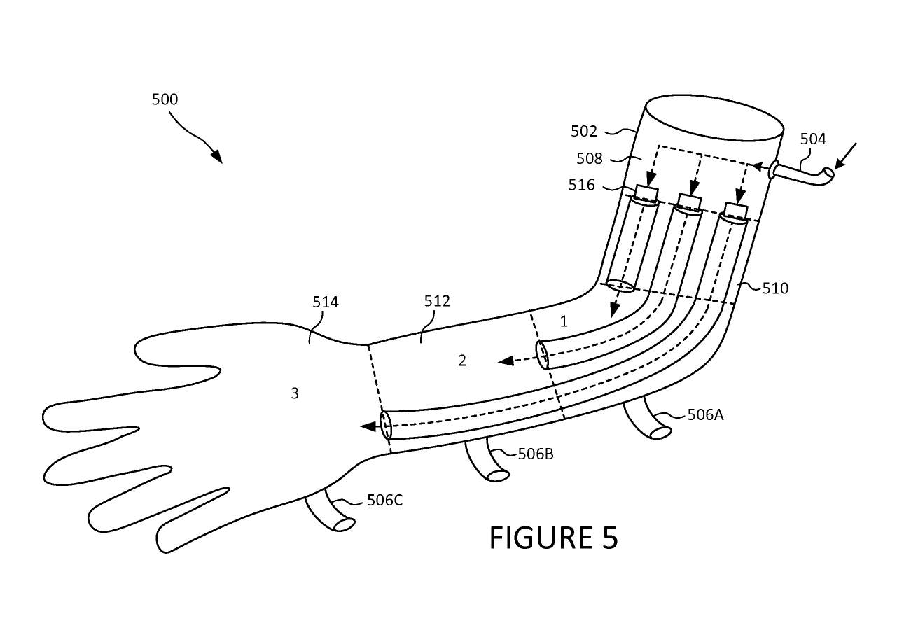

FIG. 5 shows an example compartmentalized fluid-based heat exchanging boot usable to cool or heat targeted body parts. In various embodiments, heat exchange apparatus 500 includes a single-walled or double-walled article of clothing like glove 502 with a fluid inlet 504, a number of fluid outlets 506a, 506b, and 506c, each outlet belonging to a corresponding compartment 510, 512, and 514, respectively. Each compartment connected to an inlet enclosure 508 via a separate fluid pathway or tube, shown with dotted lines, each with an external control valve 516 to control flow to each compartment.

In various embodiments, the article of clothing, whether it is a boot, a glove, a vest, or other wearable article, water-tight separate compartments 510-514 may be deployed to only cover a specific part or section of the body, such as elbow, wrist, or fingers. Each compartment may be enabled (opened) or disabled (closed) to allow fluid in or not, respectively. For example, if an elbow is injured and it needs to be cooled to reduce swelling, then only compartment 510 is opened while the other two compartments 512 and 514 are closed to concentrate the fluid and power resources (for example, the battery charge left) on the area that needs it most.

In various embodiments, the compartmentalized configuration of the heat-exchange article of clothing may include some or all of the features described above, such as heater/cooler, pump, power source, processing unit, and the like. In such configurations, the feature may be made common to all compartments. For example, the heater/cooler or pump may be deployed in or attached to entry fluid enclosure 508, which can feed or supply heated or pressurized fluid to all the compartments based on the state of the valves 516.

Changes can be made to the claimed invention in light of the above Detailed Description. While the above description details certain embodiments of the invention and describes the best mode contemplated, no matter how detailed the above appears in text, the claimed invention can be practiced in many ways. Details of the system may vary considerably in its implementation details, while still being encompassed by the claimed invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the claimed invention to the specific embodiments disclosed in the specification, unless the above Detailed Description section explicitly defines such terms. Accordingly, the actual scope of the claimed invention encompasses not only the disclosed embodiments, but also all equivalent ways of practicing or implementing the claimed invention.

The above specification, examples, and data provide a complete description of the manufacture and use of the composition of the invention. Since many embodiments of the invention can be made without departing from the spirit and scope of the invention, the invention resides in the claims hereinafter appended. It is further understood that this disclosure is not limited to the disclosed embodiments, but is intended to cover various arrangements included within the spirit and scope of the broadest interpretation so as to encompass all such modifications and equivalent arrangements.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. An apparatus for exchanging heat with a body part, the apparatus comprising:
a water-tight article of clothing to receive a heat-exchange fluid at a temperature different from a body part temperature to exchange heat with the body part, wherein the water-tight article of clothing includes a fluid inlet and a fluid outlet.

2. The apparatus of claim 1, further comprising a heating and cooling device.

3. The apparatus of claim 1, further comprising a pump usable to circulate the heat-exchange fluid.

4. The apparatus of claim 1, wherein the water-tight article of clothing is a boot.

5. The apparatus of claim 1, wherein the water-tight article of clothing includes a water-tight fluid chamber to receive the heat-exchange fluid.

6. The apparatus of claim 1, wherein the water-tight article of clothing is made of transparent material to see floating ornamental particles inside.

7. The apparatus of claim 1, wherein the water-tight article of clothing includes a power pack to run a heating/cooling device and a pump.

8. The apparatus of claim 1, wherein the water-tight article of clothing includes at least one thermometer to monitor a temperature of the heat-exchange fluid.

9. The apparatus of claim 1, wherein the water-tight article of clothing includes a processing unit to compute and display various thermal parameters.

10. A heat exchange apparatus comprising:
a water-tight boot wearable as an article of clothing, including:
a heat-exchange fluid inlet;
a heat-exchange fluid outlet; and
a heating and/or cooling device to change a temperature of a heat-exchange fluid.

11. The apparatus of claim 10, further comprising an inlet thermometer and an outlet thermometer.

12. The apparatus of claim 10, further comprising a pump to circulate the heat-exchange fluid.

13. The apparatus of claim 10, wherein the water-tight boot is single-walled and includes a fastening strap to close an opening of the water-tight boot to create a closed enclosure inside the water-tight boot.

14. The apparatus of claim 10, wherein the water-tight is double-walled having a water-tight enclosure between two walls to receive the heat-exchange fluid.

15. The apparatus of claim 10, wherein the water-tight boot includes separable compartments, each compartment being individually sealable from other compartments.

16. The apparatus of claim 10, wherein the water-proof boot includes an AC cord to provide power to the heating and/or cooling device.

17. A method of exchanging heat with a body part, the method comprising:
wearing a water-tight heat-exchange article of clothing;
pouring a heat-exchange fluid into the water-tight heat-exchange article of clothing; and
changing a temperature of the heat-exchange fluid to a value different from the body part.

18. The method of claim 17, further comprising circulating the heat-exchange fluid using a pump.

19. The method of claim 17, wherein the changing of the temperature comprises changing the temperature using an heating/cooling device integrated with the article of clothing.

20. The method of claim 17, wherein pouring the heat-exchange fluid comprises directing the heat-exchange fluid into one of several separable compartments using corresponding valves.
